(19) United States
(12) Patent Application Publication
(10) Pub. No.: US 2013/0102858 A1
(43) Pub. Date: Apr. 25, 2013

(54) IMPLANTABLE SENSOR DEVICE AND SYSTEM

(71) Applicants: Rolf Hill, Jarfalla (SE); Torbjorn Persson, Malmo (SE); Olof Stegfeldt, Alta (SE)

(72) Inventors: Rolf Hill, Jarfalla (SE); Torbjorn Persson, Malmo (SE); Olof Stegfeldt, Alta (SE)

(73) Assignee: ST. JUDE MEDICAL AB, Jarfalla (SE)

(21) Appl. No.: 13/702,148

(22) PCT Filed: Jun. 18, 2010
(86) PCT No.: PCT/EP10/58624

§ 371 Date: Dec. 5, 2012

(51) Int. Cl. A61B 5/0205; A61B 5/00; A61B 5/145; A61B 5/01; A61B 5/11; A61B 5/0215; A61N 1/362

(52) U.S. Cl. CPC A61B 5/02055; A61B 5/0215; A61B 5/6848; A61N 1/362; A61B 5/0205; A61B 5/01; A61B 5/1118; A61B 5/14542; A61B 5/14532
USPC 600/301; 600/488; 600/483

(57) ABSTRACT

The implantable medical device for measuring pressure is disclosed. The implantable medical device is connectable to a medical lead and comprises an outer sheath and a helically shaped needle arranged at the outer sheath. A pressure sensing body having a distal part is movably arranged in the outer sheath. The pressure sensing body is arranged such that the distal part is located within the outer sheath in an initial state of the pressure sensing body, wherein the pressure sensing body is arranged to be advanced from the initial state to protrude from the outer sheath and such that it is at least partially surrounded by the helically shaped needle; and a pressure sensor arranged at or adjacent to the distal part of the pressure sensing body for sensing pressure.

### FIELD OF THE INVENTION

The present invention relates generally to implantable medical devices and more particularly to implantable sensors, such as pressure sensors.

### BACKGROUND OF THE INVENTION

There are approximately 60 million people in the U.S. with risk factors for developing chronic cardiovascular diseases, including coronary cardiac disease, valvular heart disease, congenitial heart disease, cardiomyopathy and other disorders. One approach for monitoring and treating cardiovascular disease is to implant sensors, such as pressure sensors in various chambers of the heart, or adjacent vasculature such as the pulmonary arteries or veins, for the purpose of detecting, for example, early cardiac decompensation and prevention of pulmonary congestion and edema. Pressure sensors may also be useful, for example, for controlling pacemaker rate, in particular, by chronically measuring within the heart tissue.

One particular type and method of sensor placement is known as transmural placement where the sensor device enters the desired location by perforation of the tissue wall, generally, the sensor device resides on both sides of the tissue wall and within a wall separating a body structure from the rest of the body (e.g. a wall of a blood vessel or a chamber of the heart). Sensor packages can be transmurally placed in the left atrium of the heart by a minimally invasive percutaneous catheter based procedure known as transseptal catheterization.

The environment surrounding a sensor chronically implanted into the heart is very harsh, thus, entailing that the requirements placed upon such a pressure sensor are many and hard. For example, the pressure sensor must be properly protected and hermitically sealed so as to protect the sensor from degradation by the bodily fluids. Further, the sensor cannot be constructed such that the specific geometry or components cause thrombus formation, which may be potentially life threatening if caused by a sensor placed in the left atrium or left ventricle. The sensor must be stable over time, i.e. it cannot be constructed such that a “drift” of the pressure sensor occurs caused by tissue overgrowth or some other mechanism, thus resulting in inaccurate pressure measurements. If such “drifting” measurements occur, it is often difficult, or even impossible, to properly recalibrate the pressure sensor.

In U.S. Pat. No. 5,353,800, a pressure sensor lead is disclosed including a hollow needle utilized to communicate pressure to a pressure transducer. In one embodiment, a lead body includes a torque cable from which a gauge needle extends at proximal end, which gauge needle is movably arranged within a lumen within the torque cable. A solid, coiled needle is mounted around the exterior of the distal end of the torque cable, which can be rotated into cardiac tissue by corresponding rotation of the proximal end of the torque cable. Extending through the interior of the gauge needle and out the distal end thereof is a small diameter tube. The small diameter tube serves as pressure conduit which may be coupled to an external pressure transducer. To implant the lead at a desired location, the coiled needle is first screwed into the tissue by means of the torque cable. The distal end of the gauge needle is then advanced out of the distal end of the torque cable and when the tip of the gauge needle has reached or enters the pericardial fluid, the inner pressure transmitting tube of the gauge needle can be advanced into the pericardial space for pressure measurements. The point at which the gauge needle enters the pericardial space is measured by means of impedance and the point is marked with an abrupt decrease in the impedance.

However, the pressure sensor lead according to U.S. Pat. No. 5,353,800 may not fulfill at least some of the requirements placed on a pressure sensor for chronic implantation.

Thus, there is still a need within the art for pressure sensors suitable for chronic implantation.

### SUMMARY OF THE INVENTION

An object of the present invention is to provide an improved medical device and method that are capable of fulfilling at least some of the above-mentioned needs or provide a solution to or alleviating at least some of the above-mentioned problems in the prior art.

This and other objects of the present invention are achieved by means of an implantable medical device having the features defined in the independent claims. Embodiments of the invention are characterized by the dependent claims.

According to an aspect of the present invention, there is provided an implantable medical device for measuring pressure connectable to a medical lead, comprising an outer sheath and a helically shaped needle arranged at the outer sheath. A pressure sensing body having a distal part is movably arranged in the outer sheath. The pressure sensing body is arranged such that the distal part is located within the outer sheath in an initial state of the pressure sensing body, wherein the pressure sensing body is arranged to be advanced from the initial state to protrude from the outer sheath and such that it is at least partially surrounded by the helically shaped needle; and a pressure sensor arranged at or adjacent to the distal part of the pressure sensing body for sensing pressure.

According to an embodiment of the present invention, there is provided an implantable medical device for measuring pressure connectable to a lead at a proximate end of the device, comprising an outer sheath and a helically shaped needle arranged in the outer sheath, wherein the helically shaped needle is substantially completely covered by the outer sheath at an initial state of the helically shape needle and wherein the helically shaped needle is arranged to be advanced from the initial state by a screwing motion to protrude from the outer sheath. Further, the device comprises a pressure sensing body having a distal part and being movably arranged in the outer sheath, the pressure sensing body being arranged such that it is at least partially surrounded by the helically shaped needle and such that the distal part is located within the outer sheath in an initial state of the pressure sensing body, wherein the pressure sensing body is arranged to be advanced from the initial state to protrude from the outer sheath. A pressure sensor is arranged at or adjacent to the distal part of the pressure sensing body for sensing a pressure at the distal part of the pressure sensing body.

The implantable medical device for measuring pressure according to the present invention may further include sensors and/or electrodes for pacing chambers of the heart.

One advantageous embodiment of the present invention includes a combination of a sensor for measuring a pressure, e.g. left atrial pressure, and electrodes for pacing, e.g. right atrium pacing and sensing. Hence, the pressure sensing device according to the present invention can easily be combined with sensors/electrodes for pacing. For example, the outer sheath and distal end of the outer sheath can be provided with electrodes for pacing/sensing. Hence, according to another aspect of the present invention, there is provided an implantable sensor system connectable to a lead at a distal end of the lead comprising an outer sheath. At least one electrode is located on the outer sheath for delivering pacing pulses to tissue and a helically shaped needle is arranged in the outer sheath, wherein the helically shaped needle is substantially completely covered by the outer sheath at an initial state of the helically shaped needle and wherein the helically shaped needle is arranged to be advanced from the initial state by a screwing motion to protrude from the outer sheath. Further, a pressure sensing body having a distal part is movably arranged in the outer sheath, the pressure sensing body being arranged such that the distal part is located within the outer sheath in an initial state of the pressure sensing body, wherein the pressure sensing body is arranged to be advanced from the initial state to protrude from the outer sheath. A pressure sensor is further arranged at or adjacent to the distal part of the pressure sensing body for sensing pressure.

According embodiments of the present invention, the pressure sensing system includes at least one sensor for sensing a physiological and/or hemodynamical parameter including impedance, blood temperature, heart rate, an activity level of a patient, oxygen level, or blood sugar level.

The concept of the present invention provides several advantages. For example, it is possible to implant the pressure sensor without any punching/drilling of hole in the septum and it is possible to implant the pressure sensor transeptally from the right atrium or right ventricle to the left atrium or left ventricle. Moreover, it is also possible to implant a combination of pressure sensor and sensors/electrodes for pacing. The pacing lead is placed on the septum wall in either atrium or in a ventricle. After fixation of the pacing electrode using the helically shaped needle, the pressure sensing body is fed down through the inner lumen of the outer sheath towards the septum to penetrate the septum and into the atrium or ventricle on the left side of the heart. Using the present invention, the pressure sensor can be placed fast and without any additional tools required than what is normally used for lead implantation. The sensor can easily be pushed through the myocardium for access to the left side with minimal damage to the tissue of the septum.

Transmural placement of traditional physiologic sensing devices, particularly for the measurement of cardiac chamber or vascular pressures, have a number of limitations that affect long term reliable sensing and also may promote serious complications. One area of particular concern is the placement of these devices through the walls of the heart to contact the blood contained in the left atrium or adjacent regions of the left side of the heart. The devices can, for example, activate thrombus formation (blood clots, mural thrombi) on their exposed surfaces or over adjacent injured tissue. Left-sided thrombi have the potential to embolize to arteries of the systemic circulation causing catastrophic complications such as cerebral vascular accidents (stroke) and embolic infarctions of other vital organs. The lead system and pressure sensing body according to the present invention is designed to accommodate for the long-term presence of a device in the left atrium and its attendant risk of thromboembolic events, such as stroke. In particular, the pressure sensing body, which is intended to be placed through the tissue wall such that it extends into e.g. the left atrium, is designed to minimize the risk of thromboembolic events. According to the present invention, the pressure sensing body is designed with a relatively small surface area, or, in other words, designed such that the part of the sensing body that extends or protrude into the cardiac cavity (e.g. left atrium) in which the pressure will be measured has a small surface area. This is advantageous because a smaller surface area accelerates healing and decreases the chance of clot formation on the device or adjacent injured wall. Furthermore, the pressure sensing body according to the present invention is also designed to minimize the damage to tissue during transport to the desired location for implantation but, in particular, the damage to tissue caused at the implantation.

According to embodiments of the present invention, transport or insertion of the pressure sensing device through e.g. vessels and atrium or ventricles of the heart is facilitated and damage of tissue of such vessels and atrium and ventricles can be avoided in principle due to the arrangement of the helically shaped needle and the pressure sensing body within the outer sheath in a withdrawn or unscrewed state. According to an embodiment of the present invention, the pressure sensing body comprises a tip section, a first body section having a first cross-section area; and a second body section having a second cross-section area, the second cross-section area being larger than the first cross-section area; and wherein the first and second body section are arranged such that a step is formed between the first and the second body section.

This embodiment is advantageous, for example, in transmural implantation of a pressure sensor. For example, it is possible to reliably determine how far into tissue or how far into a vessel or atrium or ventricle the first body section has penetrated since the step will require a significantly higher force to penetrate a tissue wall in comparison to the force required to penetrate tissue with the body section including the tip. Accordingly, if the pressure sensing body is advanced into tissue or into a vessel or atrium or ventricle at a constant force, the step will, when it has reached the tissue wall, stop further advancement of the pressure sensing body until an increased force is applied. The increased force must be sufficient to overcome the resistance provided by the tissue wall. Thereby, a physician can determine a position of the pressure sensing body relative the tissue wall, e.g. the endocardium. The additional force required to penetrate tissue with the second body section can be made higher or lower by increasing or decreasing, respectively, the height of the step, or, in other words, the cross-section area difference between the first and second body section.

According to embodiments of the present invention, the first and second body sections are cylinder-shaped and the first body section has a smaller diameter than the second body section.

In embodiments of the present invention, the pressure sensor is integrated in the second body section. Thereby, it is possible to reliably determine the position of the pressure sensor relative to the tissue wall (e.g. endocardium) since the step will require a significantly higher force to penetrate a tissue wall in comparison to the body section including the tip. Accordingly, if the pressure sensing body is advanced into the tissue or into a vessel or atrium or ventricle at a constant force, the step will, when it has reached the tissue wall, stop further advancement of the pressure sensing body until an increased force is applied. The increased force must be sufficient to overcome the resistance provided by the tissue wall. Thereby, a physician can determine the position of the pressure sensor relative to the tissue wall, e.g. the endocardium, and the pressure sensor can be accurately positioned relative to the tissue wall. Alternatively, the pressure sensor may be integrated into the first body section. In this case, the sensor can be accurately positioned relative to a tissue wall by advancing the pressure sensing body until the step reaches a second tissue wall. The length of the first body section, i.e. the body section in which the pressure sensor is integrated in, will determine the position of the pressure sensor relative to the tissue wall. However, it is also possible to apply an additional force to force the second body section to penetrate into the vessel, atrium or ventricle, so as to advance the pressure sensor further into the vessel, atrium or ventricle.

According to embodiments of the present invention, the distal part includes one body section having a distal end shaped so as to form a cutting edge. When the distal end has been advanced through the outer sheath and rests flush against the endocardium, the cutting edge will create an incision in the tissue when the pressure sensing body is turned. Thereafter, the pressure sensing body can easily be advanced through the incision to penetrate the tissue wall, for example through the endocardium and into the myocardium.

According to embodiments of the present invention, the step between the first and second body sections is shaped so as to form a cutting edge. This is advantageous in transmural implantation of a pressure sensor. The step entails that it easy for the physician to determine where the pressure sensing body is located relative to a first tissue wall, e.g. the endocardium, since the physician will feel when the step reaches the first tissue wall and abuts the wall by increased resistance. When the step abuts the first tissue wall, the physician can create an incision in the tissue using the cutting edge by turning the pressure sensing body. Thereafter, the pressure sensing body can easily be advanced into the tissue through the incision. When a second tissue wall on the other side is reached, the procedure can be repeated, i.e. the pressure sensing body can be turned to make an incision by the cutting edge and the pressure sensing body can be pushed though the incision and into the vessel or atrium or ventricle to accurately place the pressure sensor on a desired location relative to the second tissue wall.

According to embodiments of the present invention, the pressure sensing body comprises a threaded section arranged to mate with an inner threaded surface of the outer sheath, wherein the pressure sensing body is arranged to be advanced from the initial state to protrude from the outer sheath by a screwing motion.

According to embodiments of the present invention, the pressure sensor is used to determine a position of the pressure sensing body relative to a tissue wall by means of pressure measurements. The pressure will differ depending on whether the pressure sensor is positioned in a vessel, in left or right atrium or left or right ventricle, or located within the outer sheath. By comparing the actual measured pressure with a reference pressure it is possible to determine a location of the pressure sensing body relative to a tissue or a tissue wall, for example, endocardium. It is also possible to determine an optimum placement of the pressure sensor relative to the tissue wall. For example, when the pressure sensor is located within the outer sheath, the measured pressure will be low. When the pressure sensing body has been advanced to abut the septum in right atrium the pressure will increase and when the pressure sensing body has penetrated into the tissue such that the pressure sensor is located in tissue, the pressure will decrease again. When the pressure sensing body has penetrated the septum such that the pressure sensor is located in left atrium, the pressure will be significantly higher than the pressure in the earlier locations.

Further objects and advantages of the present invention will be discussed below by means of exemplifying embodiments.

### BRIEF DESCRIPTION OF THE DRAWINGS

Exemplifying embodiments of the invention will be described below with reference to the accompanying drawings, in which:

FIG. 1 is is a partially cut-away view of a pressure sensing device according to an embodiment of the present invention;

FIG. 2 is a schematic view of an embodiment of a pressure sensing body according to the present invention;

FIG. 3 is a schematic view of another embodiment of a pressure sensing body according to the present invention;

FIG. 4 is a schematic view of a further embodiment of a pressure sensing body according to the present invention;

FIG. 5 is a schematic view of yet another embodiment of a pressure sensing body according to the present invention;

FIG. 6 is a schematic view of another embodiment of a pressure sensing body according to the present invention;

FIG. 7 shows schematically an implantable medical device according to the present invention during an implantation procedure;

FIG. 8 shows schematically the implantable medical device in FIG. 7 at a subsequent step of the implantation procedure;

FIG. 9 shows schematically the implantable medical device in FIG. 7 at a subsequent step of the implantation procedure;

FIG. 10 shows schematically the implantable medical device in FIG. 7 at a subsequent step of the implantation procedure;

FIG. 11 shows schematically the implantable medical device in FIG. 7 at a subsequent step of the implantation procedure;

FIG. 12 shows schematically the implantable medical device in FIG. 7 at a subsequent step of the implantation procedure;

FIG. 13 shows schematically the implantable medical device in FIG. 7 at a subsequent step of the implantation procedure;

FIG. 14 shows typical pressure curves for left and right atrium, respectively;

FIG. 15 shows typical pressure curves for left and right ventricle, respectively;

FIG. 16 shows a pressure curve during a movement of the pressure sensor according to the present invention from right atrium to left atrium through septum;

FIG. 17 shows a pressure curve during a movement of the pressure sensor according to the present invention from right ventricle to left ventricle through septum;

FIG. 18 is a partially cut-away view of a pressure sensing device according to a further embodiment of the present invention;

FIG. 19 is a partially cut-away view of a pressure sensing device according to the embodiment of the present invention shown in FIG. 18 where the a pressure sensing body is shown in a projected state; and

FIG. 20 is a partially cut-away view of a pressure sensing device according to the embodiment of the present invention shown in FIG. 18 where the pressure sensing body is shown in a projected state.

### DESCRIPTION OF EXEMPLIFYING EMBODIMENTS

The following is a description of exemplifying embodiments in accordance with the present invention. This description is not to be taken in limiting sense, but is made merely for the purposes of describing the general principles of the invention. It is to be understood that other embodiments may be utilized and structural and logical changes may be made without departing from the scope of the present invention. Several embodiments of the present invention relate generally to implantable pressure sensors. However, even though particular types of pressure sensors are described herein, the present invention is not limited to pressure sensors but may include other types of physiological sensors such as, for example, blood temperature sensors. The lead system and pressure sensor according to the present invention can, for example, be used with different types of implantable medical devices such as heart stimulators including biventricular pacemakers as well as other types of cardiac stimulators such as dual chamber stimulators, implantable cardioverter defibrillators (ICDs), etc.

Below, a number of embodiments of the present invention will be described as well as procedures for attaching the pressure sensing device to cardiac tissue and to position the pressure sensor at a desired location. The procedures for positioning or placing the pressure sensor at a desired location will be described with reference to a placement of the sensor in the left atrium and penetration of the endocardium and myocardium between right and left atrium. However, the present invention is suitable for a number of transmural placements, for example, the sensor can be placed in the left ventricle via a penetration of the septum between the right and left ventricle.

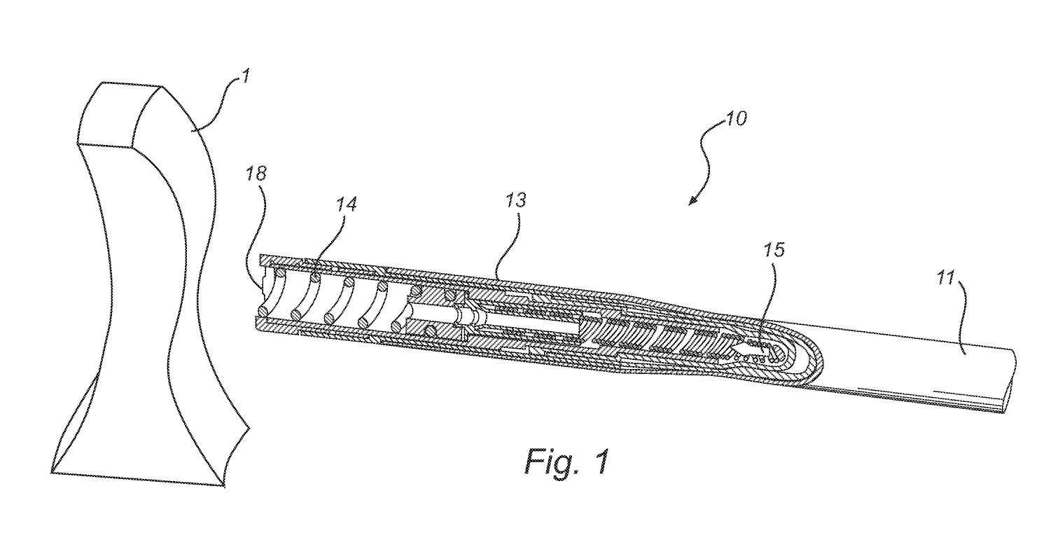

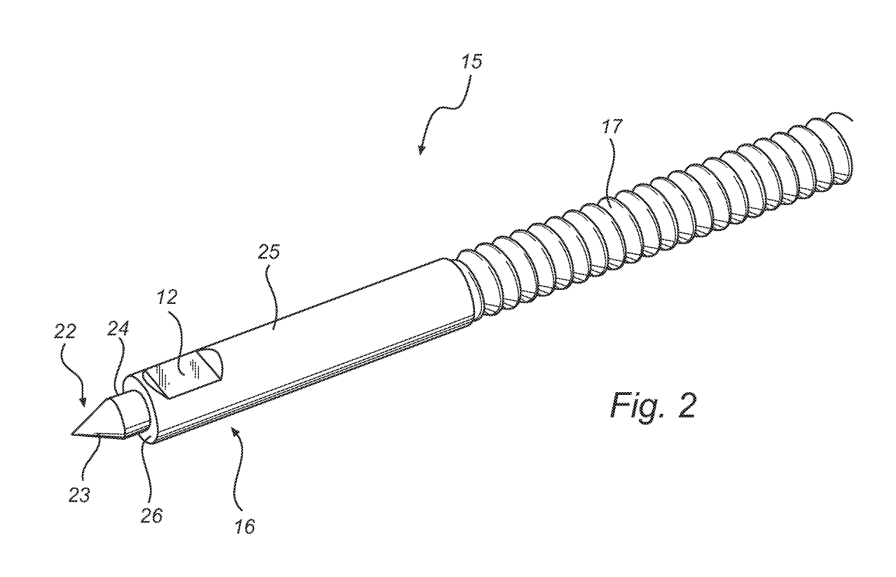

With reference now to FIG. 1, an implantable medical device according to embodiments of the present invention will be discussed. FIG. 1 is a partially cut-away view of an implantable medical device 10 for measuring pressure according to an embodiment of the present invention. According to embodiments of the present invention, the implantable medical device 10 for measuring pressure is connectable to a conventional implantable lead 11, which lead includes, for example, mutually insulated conductors (not shown) therein for carrying electrical signals between, for example, a pressure sensor 12 (see e.g. FIGS. 2-6) and circuitry in a medical device (not shown) implanted in the patient, for example, a pacemaker, or a device external to the patient.

The implantable medical device for measuring pressure according to the present invention may advantageously include, for example, electrodes for pacing a chamber of the heart, for example, right atrium. Hence, the lead may also include conductors for electrodes for delivering pacing therapy pulses to cardiac tissue and/or sensors for sensing physiological and/or hemodynamical parameters in addition to the pressure such as impedance or blood temperature.

In the following, the implantable medical device according to the present invention will be described as a pressure sensing device.

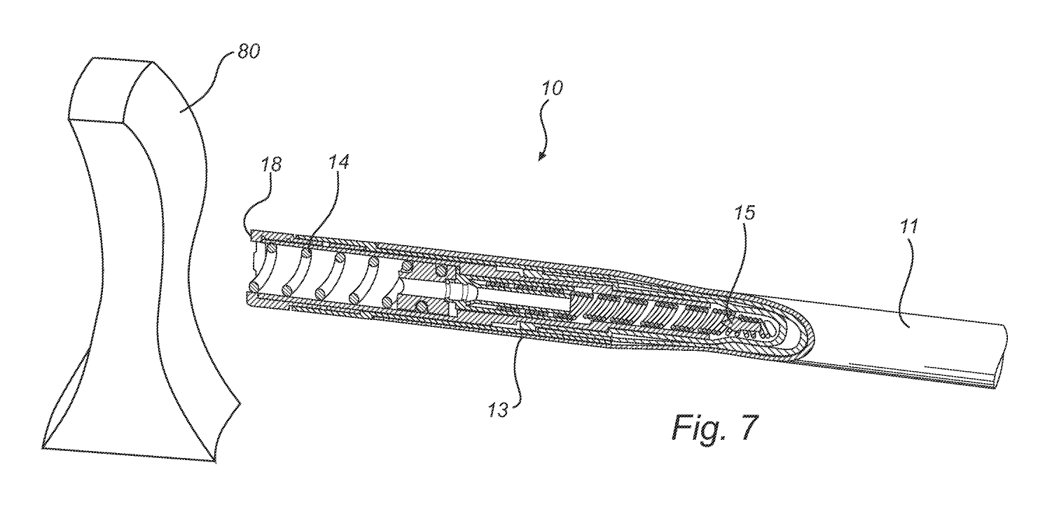

The pressure sensing device 10 comprises an outer sheath 13 and a helically shaped or coiled needle 14 arranged in the outer sheath 13. The outer sheath 13 has distal end 18 having an opening or aperture through which the helically shaped needle 14 can be advanced. In an initial state, i.e. an unscrewed state or retractile state, the helically shaped needle 14 is substantially completely covered by the outer sheath 13. The helically shaped needle 14 is arranged to be advanced from the initial state by a screwing motion to protrude from the distal end 18 of the outer sheath 13 and into cardiac tissue 1 to attach the pressure sensing system to the cardiac tissue, a procedure which is shown in FIGS. 7-13.

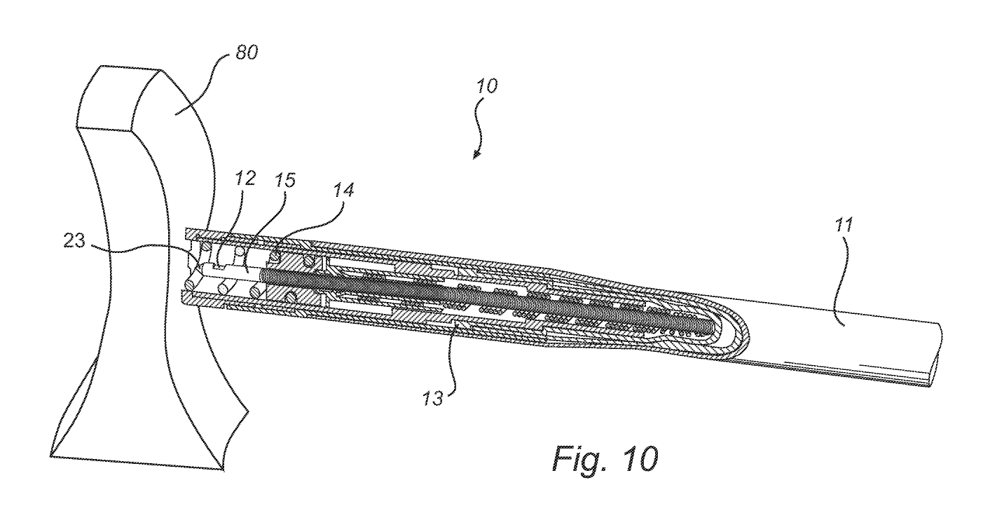

Furthermore, a pressure sensing body 15 is movably arranged in the outer sheath 13, for example, in a central lumen of the pressure sensing system 10 and lead 11. The pressure sensing body 15 is arranged such that it is at least partially surrounded by the helically shaped needle 14 and such that a distal part 16 (see FIG. 2-6) is located within the outer sheath 13 in an initial state, i.e. a retractile state, of the pressure sensing body 15, wherein the pressure sensing body 15 is arranged to be advanced from the initial state to protrude from the distal end 18 of the outer sheath 13 upon an applied force. The pressure sensing body 15 can be pushed in a linear movement to protrude from the distal end 18 of the outer sheath 13. According to embodiments, the pressure sensing body 15 is arranged to be advanced by a screwing motion. In embodiments, the pressure sensing body 15 may include a threaded section or portion 17 (see, for example, FIG. 2) arranged to mate with an inner, threaded surface of the outer sheath 13, which can be turned to advance the pressure sensing body 15 to extend from the distal end 18 of the outer sheath 13. In some embodiments, the pressure sensing body 15 is arranged to be pushed to extend from the distal end 18 of the outer sheath 13 in a linear movement and/or to be advanced in a screwing motion. For example, the pressure sensing body 15 can be pushed in a linear movement and, when placed at a desired site, the pressure sensing body 15 can be turned to place a pressure sensor 12 in a desired location (or in a desired direction) at the desired site, see e.g. FIG. 10.

The pressure sensing body 15 comprises a pressure sensor 12 integrated in or arranged at or adjacent to the distal part 16 of the pressure sensing body 15 for sensing pressure or pressure changes in an area around the distal part 16.

A suitable pressure sensor is, for example described, in U.S. RE 39,863, U.S. Pat. No. 6,248,083, or U.S. RE 35,648, which are herein incorporated by reference.

With reference to FIG. 2, a first embodiment of the pressure sensing body 15 according to the present invention will now be discussed. The pressure sensing body 15 comprises a distal part 16. In this embodiment, the distal part 16 includes a tip section 22 having a tip 23 and a first body section 24, which preferably is massive, having a first cross-section. The first cross-section has a first cross-section area. In one embodiment of the present invention, the first body section is cylinder-shaped and has a first diameter. Further, the pressure sensing body 15 comprises a second body section 25 having a second cross-section. The second cross-section has a larger cross-section area than the first body section 24. In one embodiment of the present invention, the second body section 25 is also cylinder-shaped and has a second diameter being larger than the first diameter. The first and second body sections 24 and 25 are integrally connected to form a step 26, which step 26 is formed between the first body section 24 and the second body section 25. According to the embodiment illustrated in FIG. 2, the pressure sensor 12 is integrated in the second body section 25. The second body section 25 includes a lumen or passage (not shown) so as to accommodate conductors (not shown) therein for carrying electrical signals between, for example, the pressure sensor 12 and circuitry in a medical device (not shown) implanted in the patient (or external to the patient). According to other embodiments of the present invention, the distal section 16 comprises a tip and a body section including the sensor. In this embodiment, the pressure sensing body does not include a step.

The pressure sensing body 15 further includes a threaded section or portion 17 arranged to mate with an inner, threaded tubing arranged in the outer sheath 13. Thus, by turning the pressure sensing body it can be advanced to protrude from the distal end 18 of the outer sheath 13 and penetrate cardiac tissue (e.g. the endocardium), which will be illustrated below with reference to FIGS. 7-13. Alternatively, the pressure sensing body can be advanced through the distal end 18 of the outer sheath 13 in a sliding motion to protrude from the outer sheath 13 by applying a force on the pressure sensing body 15. In operation, the pressure sensing body 15 can be, when the pressure sensing device has been properly attached to cardiac tissue by means of the helically shaped needle 14, advanced to protrude from the distal end 18 of the outer sheath 13. When attached to cardiac tissue the open distal end of the outer sheath 13 rests flush against the cardiac tissue. The pressure sensing body 15 will penetrate the cardiac tissue, e.g. the endocardium, when the tip 23 reaches the endocardium and the step 26 entails that it easy for the operator, for example, the physician to know where the pressure sensing body 15 is relative to the tissue. That is, the physician feels when the step 26 reaches the endocardium wall by an increased resistance. By applying an additional force, the pressure sensing body 15 will penetrate further into the endocardium, i.e. the second body part 25 will also penetrate the endocardium wall. It is possible to vary the additional force required to penetrate the endocardium with also the second body portion 25 by making the step larger, or in other words, by making the cross-section area difference bigger. Hence, the smaller the step 26 is made, the smaller the additional force required will be, and similarly, a larger the step requires a larger additional force. The pressure sensing body 15 can thus be advanced to penetrate into the myocardium and through the endocardium wall on the left side. When the step 26 reaches the endocardium wall on the left side the physician will similarly feel that by an increased resistance and likewise to the entry into the myocardium by the second body section 25, an additional force is required to push the second body section 24 through the endocardium wall and, thereby, place the pressure sensor 12 on a desired location relative to the endocardium wall. As mentioned above, this will be described below with reference to FIGS. 7-13.

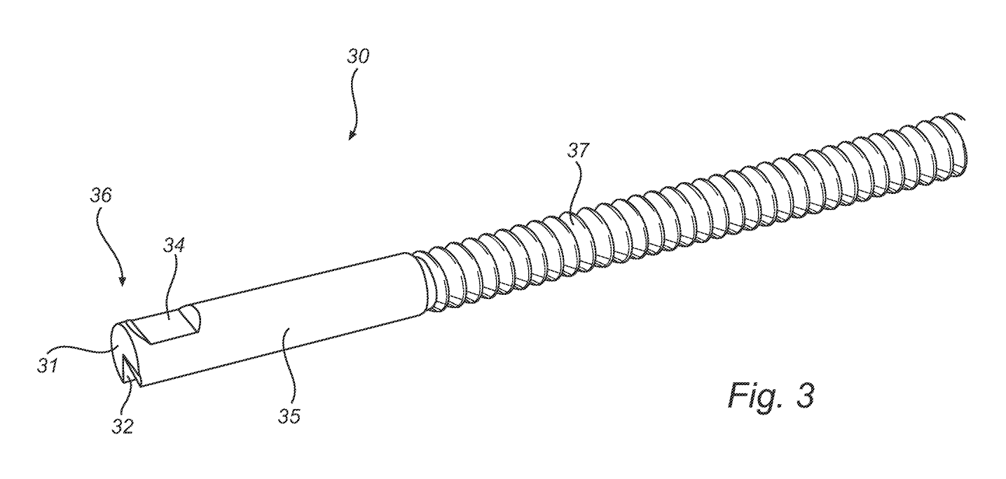

With reference now to FIG. 3, a further embodiment of the present invention will be discussed. The pressure sensing body 30 comprises a distal part 36. including a body section 35. In this embodiment, a distal end 31 of the body section 35 includes a cutting edge 32. In a preferred embodiment, the body section 35 is cylinder-shaped. A pressure sensor 34 is integrated in the body section 35. The body section 35 includes a lumen or passage (not shown) so as to accommodate conductors (not shown) therein for carrying electrical signal between, for example, the pressure sensor 34 and circuitry in a medical device (not shown) implanted in the patient (or external to the patient). The pressure sensing body 30 further includes a threaded section or portion 37 arranged to mate with an inner, threaded tubing arranged in the outer sheath 13. Thus, by turning the pressure sensing body it can be advanced to protrude from the outer sheath 13 and penetrate cardiac tissue (e.g. the endocardium), which will be illustrated below with reference to FIGS. 7-13. Alternatively, the pressure sensing body can be advanced through the outer sheath 13 in a sliding motion to protrude from the distal end 18 of the outer sheath 13 by applying a force on the pressure sensing body 30. In operation, the pressure sensing body 30 can be, when the pressure sensing device has been properly attached to cardiac tissue by means of the helically shaped needle 14, advanced to protrude from the distal end of the outer sheath 13. By advancing the pressure sensing body 30 until the distal end 31 rests flush against the endocardium and then turning the pressure sensing body 30, the cutting edge 32 will create an incision in the tissue. Thereafter, the pressure sensing body 30 can be advanced through the incision to penetrate into the myocardium. When the endocardium is reached on the left side, the procedure can be repeated, i.e. the pressure sensing body can be turned to make an incision by the cutting edge 32, the pressure sensing body can be pushed though the incision and into the left atrium to place the pressure sensor 34 on a desired location relative to the endocardium wall, as will be described below with reference to FIGS. 7-13.

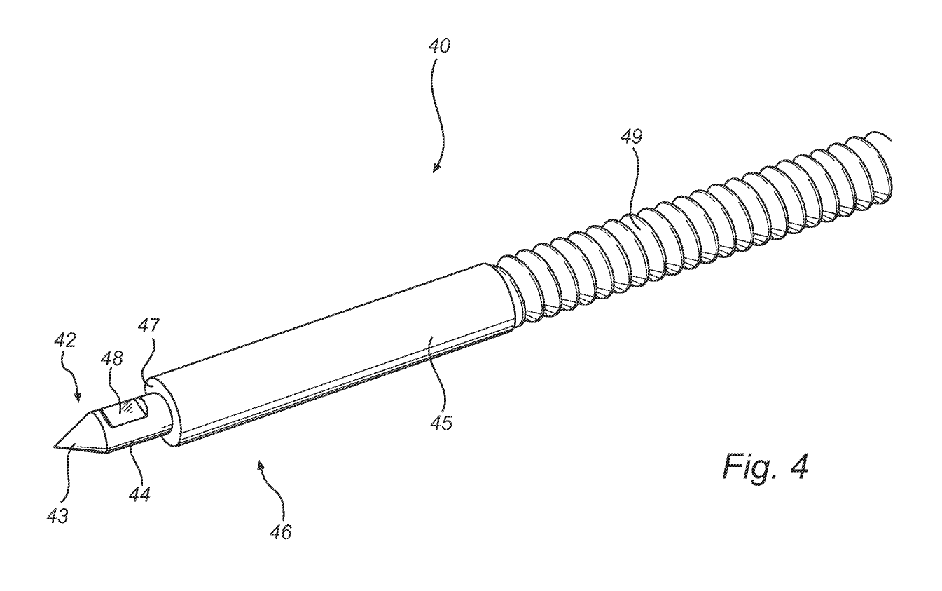

With reference to FIG. 4, another embodiment of the pressure sensing body according to the present invention will be discussed. The pressure sensing body 40 comprises a distal part 46. In this embodiment, the distal part 46 includes a tip section 42 having a tip 43, which preferably is massive, and a first body section 44 having a first cross-section. The first cross-section has a first cross-section area. In one embodiment of the present invention, the first body section is cylinder-shaped and has a first diameter. Further, the pressure sensing body 40 comprises a second body section 45 having a second cross-section. The second cross-section has a larger cross-section area than the first body section 44. In one embodiment of the present invention, the second body section 45 is cylinder-shaped and has a second diameter being larger than the first diameter. The first and second body sections 44 and 45 are integrally connected to form a step 47, which step 47 is formed between the first body section 44 and the second body section 45. According to the embodiment illustrated in FIG. 2, the pressure sensor 48 is integrated in the first body section 44. The first and second body section 44, 45 includes a lumen or passage (not shown) so as to accommodate conductors (not shown) therein for carrying electrical signal between, for example, the pressure sensor 48 and circuitry in a medical device (not shown) implanted in the patient (or external to the patient). The pressure sensing body 40 further includes a threaded section or portion 49 arranged to mate with an inner, threaded tubing arranged in the outer sheath 13. Thus, by turning the pressure sensing body it can be advanced to protrude from the distal end 18 of the outer sheath 13 and penetrate cardiac tissue (e.g. the endocardium), which will be illustrated below with reference to FIGS. 7-13. Alternatively, the pressure sensing body can be advanced through the distal end 18 of the outer sheath 13 in a sliding motion to protrude from the outer sheath 13 by applying a force on the pressure sensing body 40. In operation, the pressure sensing body 40 can be, when the pressure sensing device has been properly attached to cardiac tissue by means of the helically shaped needle 14, advanced to protrude from distal end 18 of the outer sheath 13. When attached to cardiac tissue, the distal end 18 of the outer sheath 13 rests flush against the cardiac tissue. The pressure sensing body 40 will penetrate the cardiac tissue, e.g. the endocardium, when the tip 43 reaches the endocardium and the step 47 entails that it easy for the operator, for example, the physician to determine where the pressure sensing body 40 is located relative to the tissue wall. That is, the physician feels when the step 47 reaches the endocardium wall as an increased resistance. By applying an additional force, the pressure sensing body 45 will penetrate into the myocardium, i.e. the second body part 25 will also penetrate the endocardium. It is possible to vary the additional force required to penetrate the endocardium with also the second body portion 45 by making the step larger, or in other words, by making the cross-section area difference bigger. Hence, the smaller the step 47 is made, the smaller the additional force required will be, and similarly, a larger the step requires a larger additional force. When the endocardium on the left side has been reached by the tip 43, it will penetrate the endocardium into the left atrium, and the pressure sensor 48 will thereby be placed in the left atrium. The step 47 entails that the physician will fell when the pressure sensing body 15 has entered into the left atrium and thus when the pressure sensor 48 has been placed in the desired location relative to the endocardium. In this case, the physician will not apply an increased or additional force so as to penetrate the endocardium with also the second body section 45.

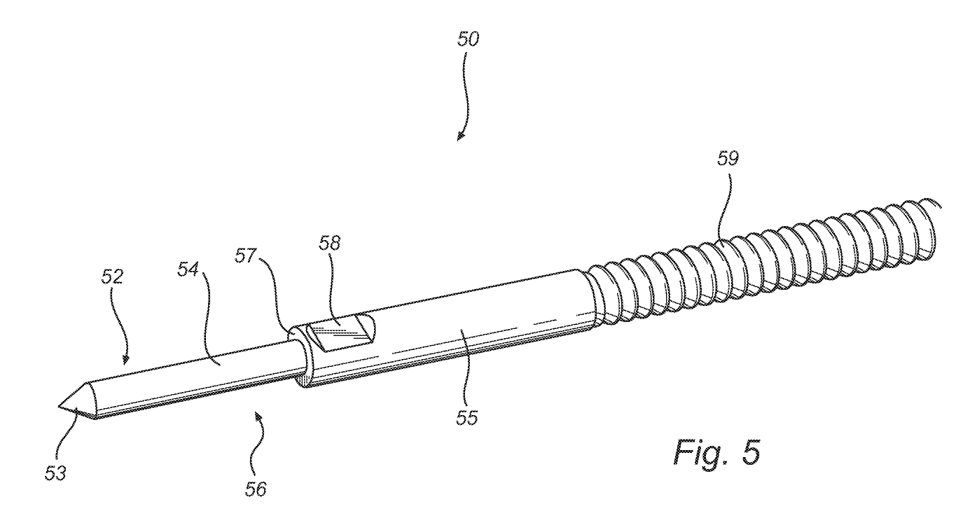

Yet another embodiment of the present invention will now be discussed with reference to FIG. 5. The pressure sensing body 50 comprises a distal part 56. In this embodiment, the distal part 56 includes a tip section 52 having a tip 53 and a first body section 54, which preferably is massive, having a first cross-section. The first cross-section has a first cross-section area. In one embodiment of the present invention, the first body section is cylinder-shaped and has a first diameter. Further, the pressure sensing body 50 comprises a second body section 55 having a second cross-section. The second cross-section has a larger cross-section area than the first body section 54. In one embodiment of the present invention, the second body section 55 is cylinder-shaped and has a second diameter being larger than the first diameter. The first and second body sections 54 and 55 are integrally connected to form a step 57, which step 57 is formed between the first body section 54 and the second body section 55. In this embodiment, the first body section 54 is considerably longer than the first body section 24 of the embodiment illustrated in FIG. 2, which may be an advantage if, for example, a thicker part of septum is intended to be penetrated.

According to the embodiment illustrated in FIG. 5, the pressure sensor 58 is integrated in the second body section 55. The second body section 55 includes a lumen or passage (not shown) so as to accommodate conductors (not shown) therein for carrying electrical signal between, for example, the pressure sensor 58 and circuitry in a medical device (not shown) implanted in the patient (or external to the patient). The pressure sensing body 50 further includes a threaded section or portion 59 arranged to mate with an inner, threaded tubing arranged in the outer sheath 13. Thus, by turning the pressure sensing body it can be advanced to protrude from the distal end 18 of the outer sheath 13 and penetrate cardiac tissue (e.g. the endocardium), which will be illustrated below with reference to FIGS. 7-13. Alternatively, the pressure sensing body can be advanced through the distal end 18 of the outer sheath 13 in a sliding motion to protrude from the outer sheath 13 by applying a force on the pressure sensing body 50. In operation, the pressure sensing body 50 can be, when the pressure sensing device has been properly attached to cardiac tissue by means of the helically shaped needle 14, advanced to protrude from the distal end 18 of the outer sheath 13. When attached to cardiac tissue the open distal end of the outer sheath 13 rests flush against the cardiac tissue. The pressure sensing body 50 will penetrate the cardiac tissue, e.g. the endocardium, when the tip 53 reaches the endocardium and the step 57 entails that it easy for the operator, for example, the physician to know where the pressure sensing body 50 is relative to the tissue wall. That is, the physician will feel when the step 57 reaches the endocardium by an increased resistance. By applying an additional force to the force used to advance the first body section 54 through the tissue on the pressure sensing body 50, the pressure sensing body 50 will penetrate further into the myocardium, i.e. the second body part 55 will also penetrate the endocardium wall. It is possible to vary the additional force required to penetrate the endocardium with also the second body portion 55 by making the step larger, or in other words, by making the cross-section area difference bigger. Hence, the smaller the step 57 is made, the smaller the additional force required will be, and similarly, a larger the step requires a larger additional force. The pressure sensing body 50 can thus be advanced to penetrate into the myocardium and through the endocardium wall on the left side. When the step 57 reaches the endocardium wall on the left side the physician will similarly feel that by an increased resistance and likewise to the entry into the myocardium by the second body section 55, an additional force is required to push the second body section 55 through the endocardium wall and, thereby, place the pressure sensor 58 on a desired location relative to the endocardium wall. As mentioned above, this will be described below with reference to FIGS. 7-13.

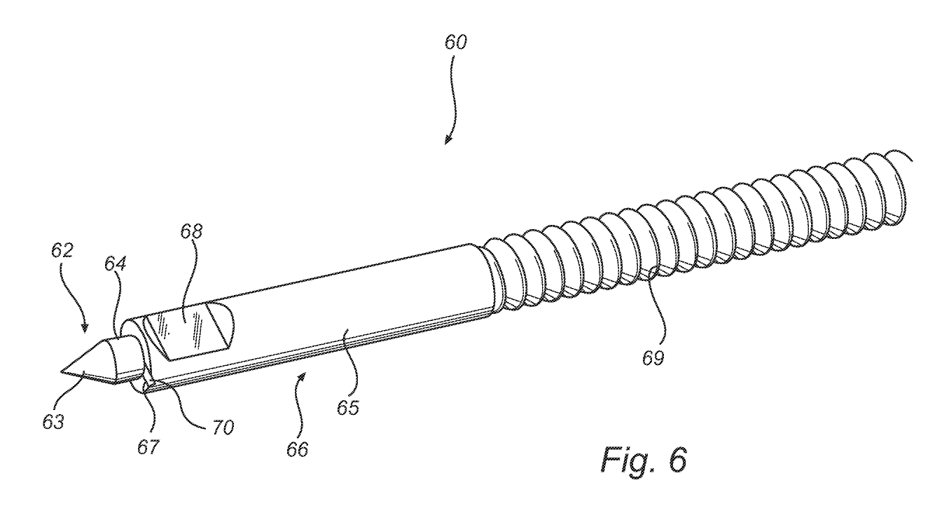

Another embodiment of the present invention will now be discussed with reference to FIG. 6. The pressure sensing body 60 comprises a distal part 66. In this embodiment, the distal part 66 includes a tip section 62 having a tip 63 and a first body section 64, which preferably is massive, having a first cross-section. The first cross-section has a first cross-section area. In one embodiment of the present invention, the first body section is cylinder-shaped and has a first diameter. Further, the pressure sensing body 60 comprises a second body section 65 having a second cross-section. The second cross-section has a larger cross-section area than the first body section 64. In one embodiment of the present invention, the second body section 65 is cylinder shaped and has a second diameter being larger than the first diameter. The first and second body sections 64 and 65 are integrally connected to form a step 67, which step 67 is formed between the first body section 64 and the second body section 65. In this embodiment, the step 67 is arranged to form a cutting edge 70. Furthermore, a pressure sensor 68 is integrated in the second body section 65. The second body section 65 includes a lumen or passage (not shown) so as to accommodate conductors (not shown) therein for carrying electrical signal between, for example, the pressure sensor 12 and circuitry in a medical device (not shown) implanted in the patient (or external to the patient). The pressure sensing body 60 further includes a threaded section or portion 69 arranged to mate with an inner, threaded tubing arranged in the outer sheath 13. Thus, by turning the pressure sensing body 60, it can be advanced to protrude from the distal end 18 of the outer sheath 13 and penetrate cardiac tissue (e.g. the endocardium), which will be illustrated below with reference to FIGS. 7-13. Alternatively, the pressure sensing body 60 can be advanced through the distal end 18 of the outer sheath 13 in a sliding motion to protrude from the outer sheath 13 by applying a force on the pressure sensing body 60. In operation, the pressure sensing body 60 can be, when the pressure sensing device has been properly attached to cardiac tissue by means of the helically shaped needle 14, advanced to protrude from the distal end 18 of the outer sheath 13. When attached to cardiac tissue the open distal end of the outer sheath 13 rests flush against the cardiac tissue. The step 67 entails that it easy for the operator, for example, a physician to determine where the pressure sensing body 60 is located relative to a tissue wall. That is, the physician is able to feel when the step 67 reaches e.g. endocardium by an increased resistance. When the step 67 rests flush against the endoracdium wall, the physician can by turning the pressure sensing body 60 create an incision in the tissue using the cutting edge 70 arranged in the step 67. Thereafter, the pressure sensing body 60 can be advanced into the myocardium through the incision. When the endocarium is reached on the left side, the procedure can be repeated, i.e. the pressure sensing body can be turned to make an incision by the cutting edge 70, the pressure sensing body can be pushed through the incision and into the left atrium to place the pressure sensor 68 on a desired location relative to the endocardium.

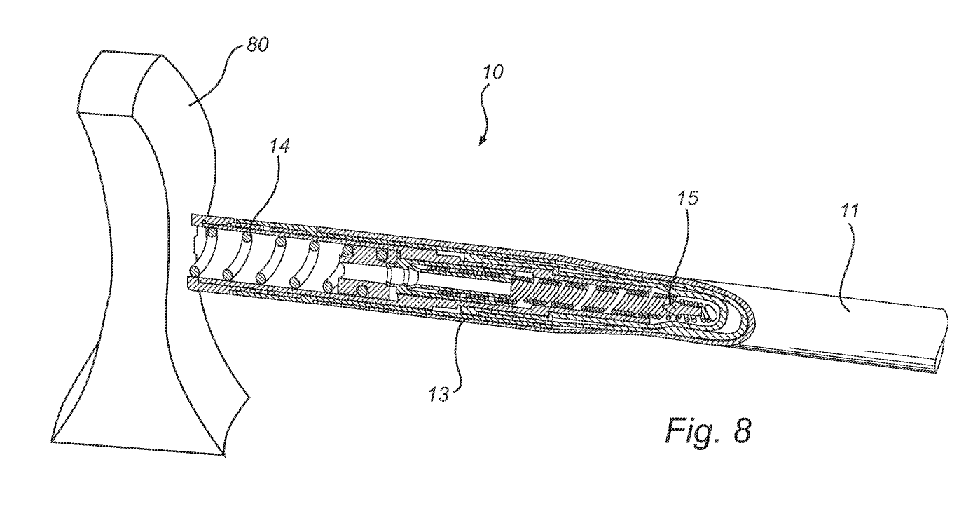

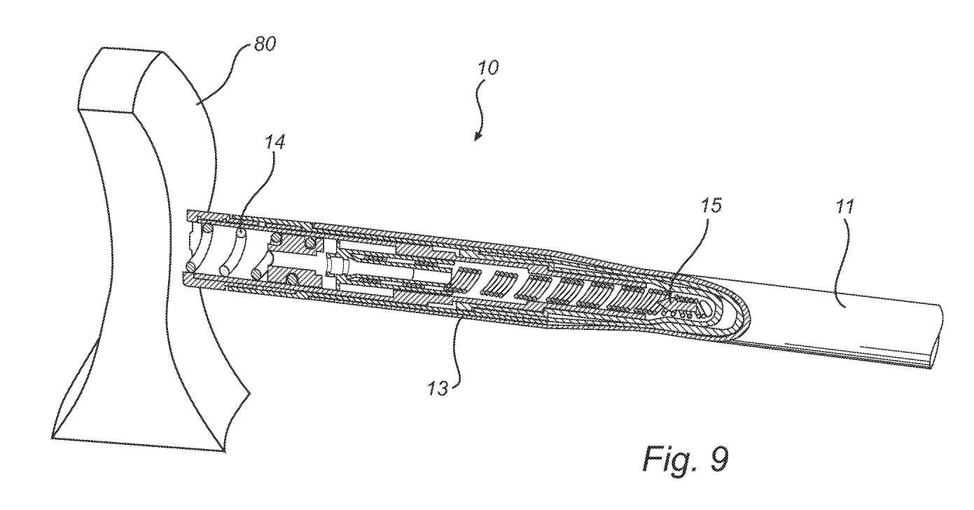

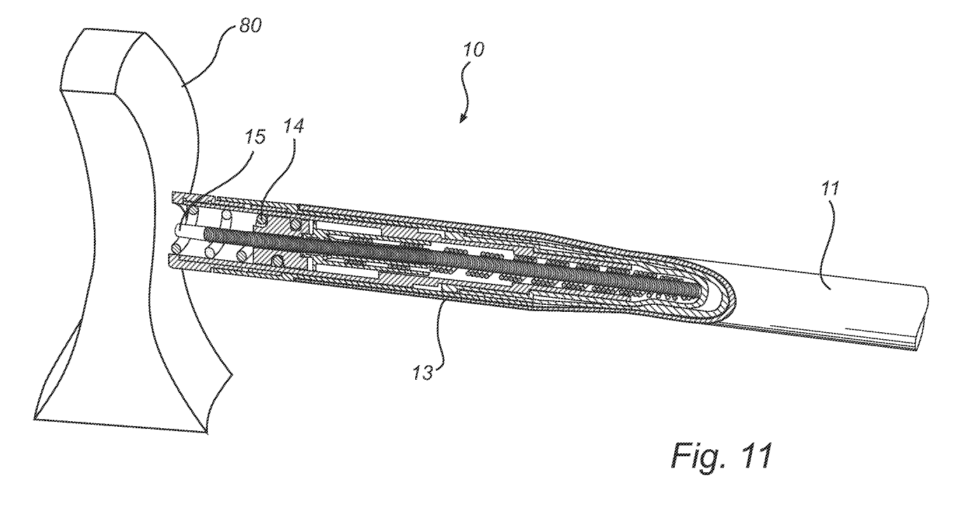

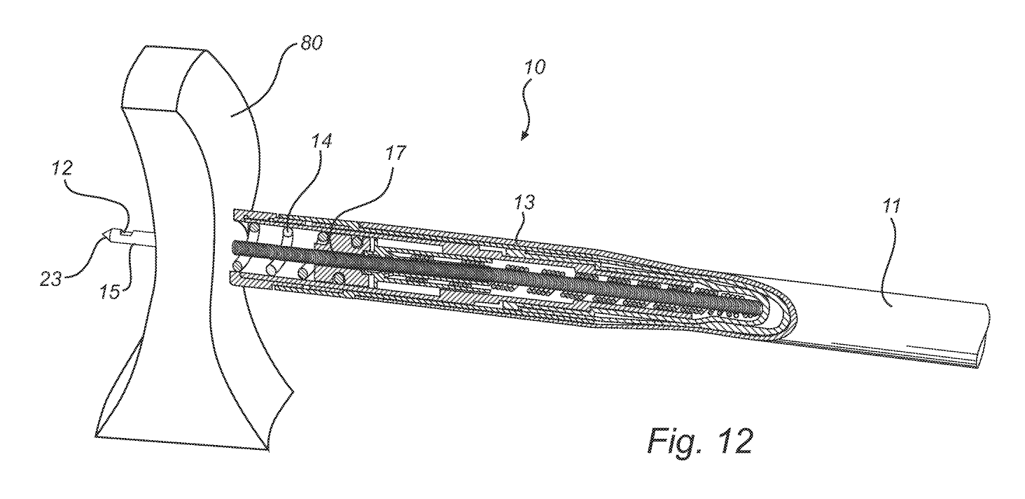

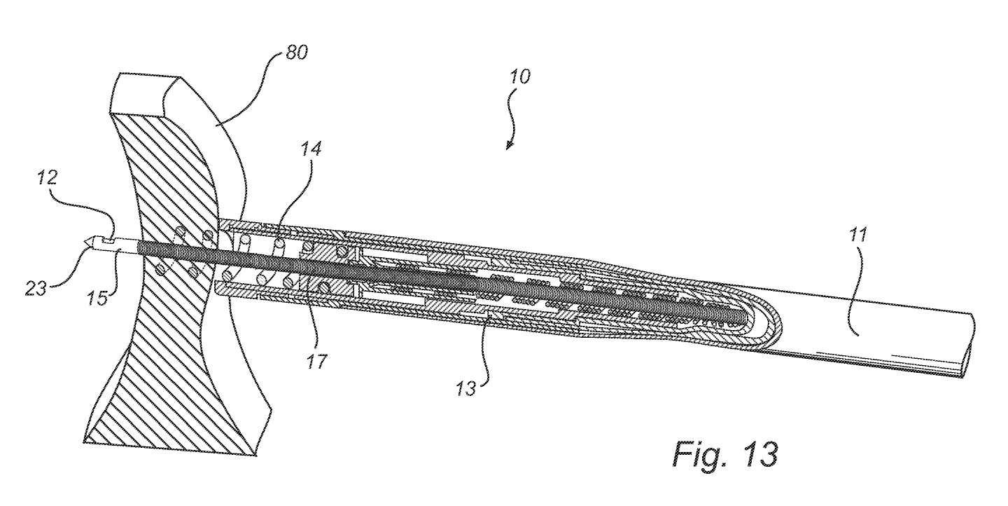

With reference now to FIG. 7-13, a procedure for placing a pressure sensor of a pressure sensing device according to the present invention will be discussed. In order to exemplify, the procedure will be described with reference to the embodiment of the pressure sensing device described in FIGS. 1 and 2. However, as the skilled person realizes, any one of the embodiments described herein is suitable for use in the procedure described with reference to FIG. 7-13. As can be seen in FIG. 7, the pressure sensing device 10 is moved towards a tissue wall 80 of the heart, for example, endocardium in the right atrium. During movement of the pressure sensing device 10 in, for example, a blood vessel, the helically shaped needle 14 and the pressure sensing body 15 are in a withdrawn or retracted state within the outer sheath 13 to inter alia protect tissue and to facilitate movement of the pressure sensing device 10. When the pressure sensing device 10 has reached the endocardium 80 at a desired position, the helically shaped needle 14 is screwed into the endocardium 80 to fixate the pressure sensing device 10 to the endocardium at the desired position, see FIGS. 8 and 9. When the pressure sensing device 10 has been fixated to the endocardium, the pressure sensing body 15 can be advanced through the outer sheath 13 to reach the endocardium 80, as shown in FIG. 10. Thereafter, the tip 23 of the pressure sensing body 15 can penetrate the endocardium 80 and the pressure sensing body can be advanced into the myocardium, see FIG. 11. In FIG. 12, it is shown how the pressure sensor 12 has reached the left atrium, i.e. the pressure sensing body 15 has now penetrated into the left atrium. At this position, the pressure sensing device 15 is now fixated by means of the helically shaped needle 14 and it is possible to chronically measure the pressure in left atrium. In FIG. 13, a cross-section of the endocardium and the myocardium are shown to illustrate how the pressure sensing body 15 and the helically shaped needle 14 cooperates to fixate the pressure sensing device 10 to the endocardium 80 and to position the pressure sensor 12 in the left atrium.

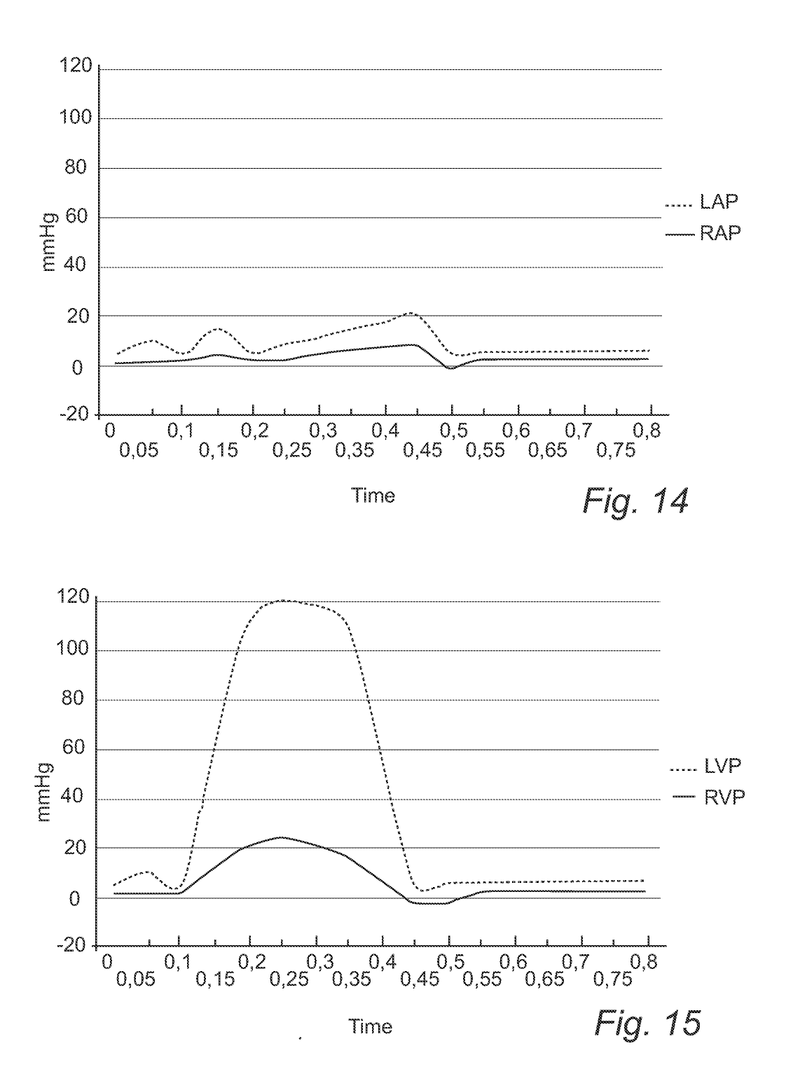

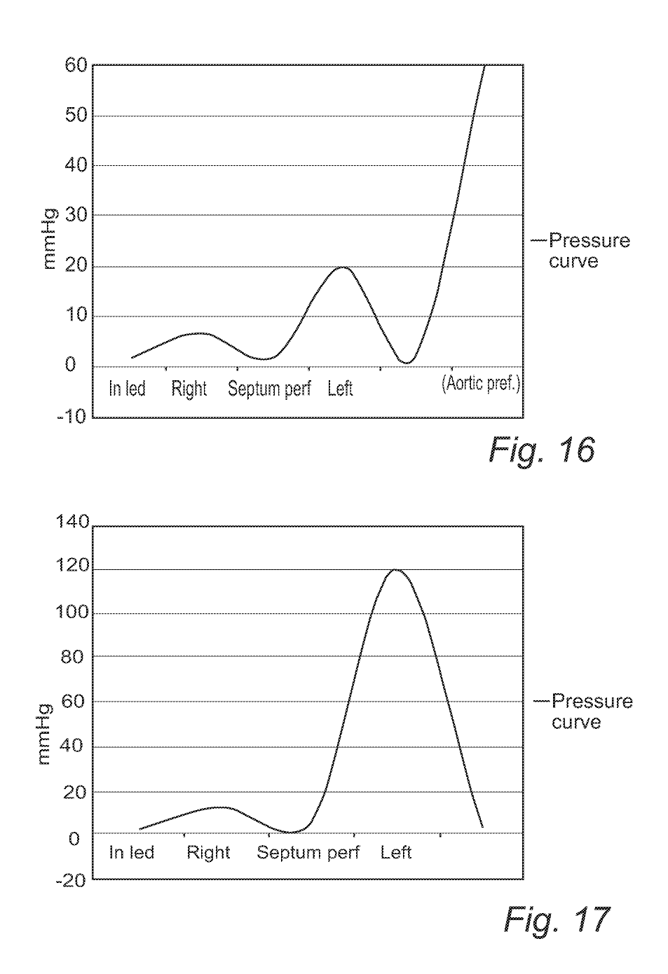

According to embodiments of the present invention, the pressure sensor is used to determine a position of the pressure sensing body relatively a tissue wall by means of pressure measurements. The measured pressure will be different depending on whether the pressure sensor is positioned in, for example, a vessel, in left or right atrium or left or right ventricle, located in tissue or located within the outer sheath. By comparing the measured pressure with a reference pressure it is possible to determine a location of the pressure sensing body relative to a tissue or a tissue wall, for example, endocardium. It may also be possible to determine an optimum placement of the pressure sensor relative to the tissue wall by using the measured pressure. In FIGS. 14 and 15, typical pressure curves for left and right atrium and left and right ventricle are shown during a cardiac cycle, respectively. Further, in FIG. 16, the pressure variation when the pressure sensor is advanced from an initial position within the outer sheath, via right atrium, through the septum and into a position within the left atrium and further to a location with an aortic perforation is shown. In FIG. 17 the pressure variation when the pressure sensor is advanced from an initial position within the outer sheath, via right ventricle, through the septum and into a position within the left ventricle is shown.

It should be noted that the pressures measured at different sensor positions shown in FIGS. 16 and 17 are measured at time point of the cardiac cycle where the systolic pressure reaches the highest value.

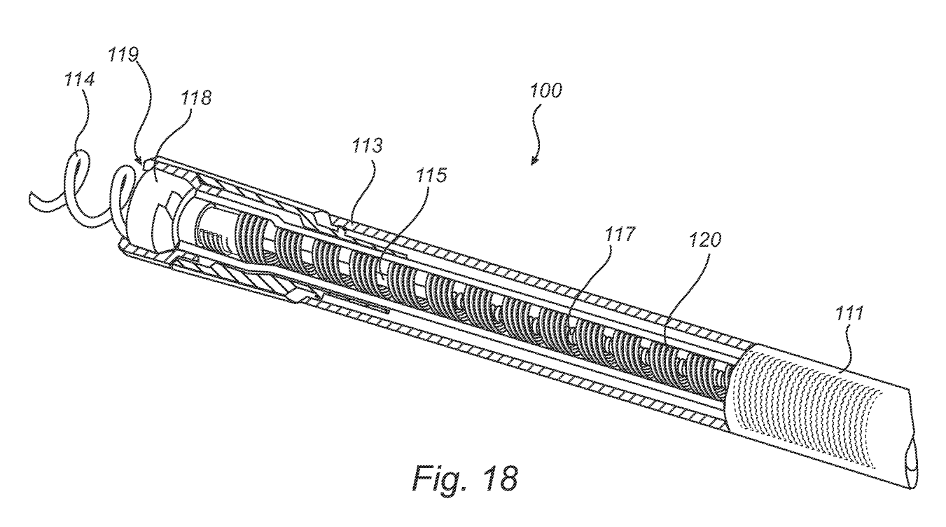

With reference now to FIGS. 18-20, a further embodiment of the present invention will be discussed. FIG. 18 is a partially cut-away view of an implantable medical device 100 for measuring pressure according to an embodiment of the present invention. According to embodiments of the present invention, the implantable medical device 100 for measuring pressure, which in the following will be described as a pressure sensing device, is connectable to a conventional implantable lead 111, which lead includes, for example, mutually insulated conductors (not shown) therein for carrying electrical signals between, for example, a pressure sensor 112 and circuitry in a medical device (not shown) implanted in the patient, for example, a pacemaker, or a device external to the patient.

The implantable medical device for measuring pressure according to this embodiment of the present invention may advantageously include, for example, electrodes for pacing a chamber of the heart, for example, right atrium. Hence, the lead may also include conductors for electrodes for delivering pacing therapy pulses to cardiac tissue and/or sensors for sensing physiological and/or hemodynamical parameters in addition to the pressure such as impedance or blood temperature.

In the following, the implantable medical device according to the present invention will be described as a pressure sensing device.

The pressure sensing device 100 comprises an outer sheath 113 and a helically shaped or coiled needle 114 fixated at a distal element 118 fitted in a distal opening 119 of the outer sheath 113. The distal element 118 may be suited with a steroid plug or contain a steroid plug. A steroid plug may alternatively be arranged at the outer sheath 113 as a collar.

The helically shaped needle 114 is arranged to be screwed or rotated into cardiac tissue to attach the pressure sensing device 100 to the cardiac tissue by corresponding rotation of the outer sheath 113. A similar procedure is shown in FIGS. 7-13, with the difference that the embodiment shown in FIGS. 7-13 has a movable helically shaped coil in contrast to the embodiment shown in FIGS. 18-20 which has a fixed helically shaped needle. Hence, the pressure sensing device is attached to cardiac tissue by means screwing the helically shaped needle into the tissue either by rotation of the helically shaped needle or by rotating of the outer sheath and thereby a corresponding rotation of the helically shaped needle.

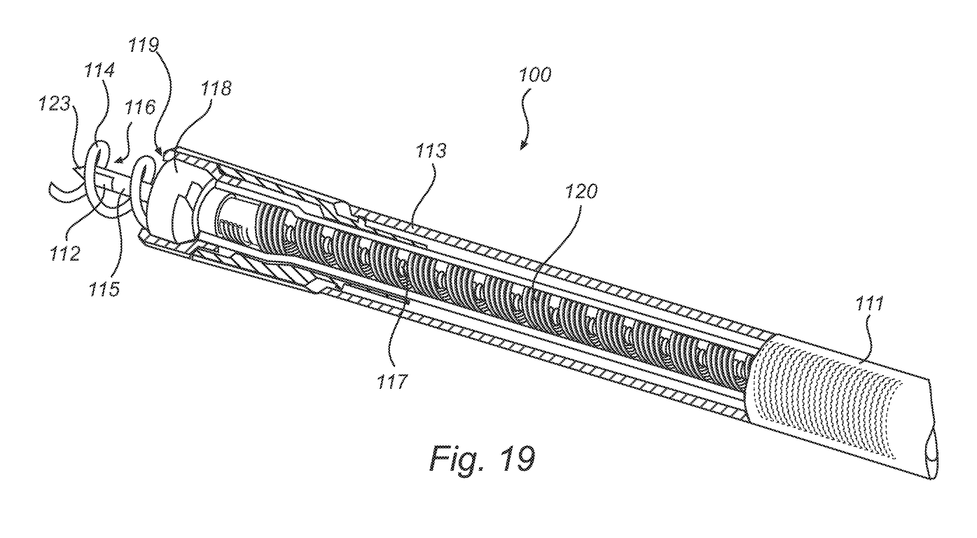

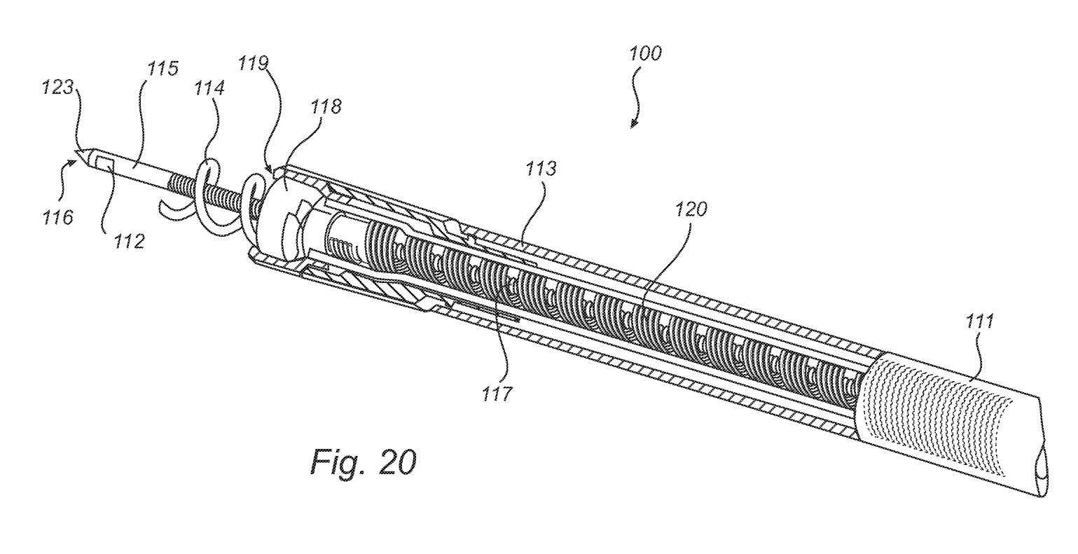

Furthermore, a pressure sensing body 115 is movably arranged in the outer sheath 113, for example, in a central lumen of the outer sheath 113 and lead 111. The pressure sensing body 115 is arranged such that the pressure sensor 112 and a distal tip 123 (see FIG. 19-20) is located within the outer sheath 113 in an initial state, i.e. a retractile state, of the pressure sensing body 115, wherein the pressure sensing body 115 is arranged to be advanced from the initial state to protrude through the distal element 118 of the outer sheath 113 upon an applied force. The pressure sensing body 115 can be pushed in a linear movement to protrude from the distal end 118 of the outer sheath 113. According to embodiments, the pressure sensing body 115 is arranged to be advanced by a screwing motion. In embodiments, the pressure sensing body 115 may include a threaded section or portion 117 arranged to mate with an inner, threaded tubing 120 arranged in the outer sheath 113, which can be turned to advance the pressure sensing body 115 to extend through the distal element 118 of the outer sheath 113. In some embodiments, the pressure sensing body 115 is arranged to be pushed to extend through the distal element 118 of the outer sheath 113 in a linear movement and/or to be advanced in a screwing motion. For example, the pressure sensing body 115 can be pushed in a linear movement and, when placed at a desired site, the pressure sensing body 115 can be turned to place a pressure sensor 112 in a desired location (or in a desired direction) at the desired site. In FIGS. 19 and 20, the pressure sensing body 115 is shown in states where the pressure sensing body 115 have been advanced out from the outer sheath 113.

The pressure sensing body 115 comprises a pressure sensor 112 integrated in or arranged at or adjacent to a distal part 116 of the pressure sensing body 115 for sensing pressure or pressure changes in an area around the distal part 116. A suitable pressure sensor is, for example described, in U.S. RE 39,863, U.S. Pat. No. 6,248,083, or U.S. RE 35,648, herein incorporated by reference.

A pressure sensing body in accordance with any one of the embodiments described with reference to FIGS. 2-6 can be used in the embodiment of the pressure sensing device described with reference to FIGS. 18-20.

Although certain embodiments and examples have been described herein, it will be understood by those skilled in the art that many aspects of the devices and methods shown and described in the present disclosure may be differently combined and/or modified to form still further embodiments. Alternative embodiments and/or uses of the devices and methods described above and obvious modifications and equivalents thereof are intended to be within the scope of the present disclosure. Thus, it is intended that the scope of the present invention should not be limited by the particular embodiments described above, but should be determined by a fair reading of the claims that follow.

Additionally, the skilled artisan will recognize that the embodiments of the pressure sensing system and pressure sensing body described herein may advantageously be applied for implanting pressure sensors transmurally on, in or through a wall of any organ or vessel within a patient. It will also be apparent to one skilled in the art that the field of use of the embodiments of the pressure sensing system and pressure sensing body described herein extends beyond the specific conditions of measuring the pressure in left atrium to measurements of pressure where the pressure sensor is implanted through a wall of a chamber or a vessel or is positioned approximate to a wall of that chamber or vessel.

1-15. (canceled)

16. An implantable medical device for measuring pressure connectable to a medical lead, comprising:
an outer sheath;
a helically shaped needle arranged at said outer sheath;
a pressure sensing body having a distal part and being movably arranged in said outer sheath, said pressure sensing body being arranged such that said distal part is located within said outer sheath in an initial state of said pressure sensing body, wherein said pressure sensing body is arranged to be advanced from said initial state to protrude from said outer sheath such that it is at least partially surrounded by said helically shaped needle; and
a pressure sensor arranged at or adjacent to said distal part of said pressure sensing body for sensing pressure.

17. The implantable medical device according to claim 16, wherein said helically shaped needle is fixed at said outer sheath or at a distal element fitted in a distal opening of said outer sheath.

18. The implantable medical device according to claim 17, wherein said distal element comprises a through hole arranged such that said pressure sensing body can be advanced through said through hole.

19. The implantable medical device for measuring pressure connectable to a medical lead according to claim 16, wherein
said helically shaped needle is substantially completely covered by said outer sheath at an initial state of said helically shape needle and wherein said helically shaped needle is arranged to be advanced from said initial state by a screwing motion to protrude from said outer sheath;
said pressure sensing body being arranged such that it is at least partially surrounded by said helically shaped needle and such that said distal part is located within said outer sheath in an initial state of said pressure sensing body, wherein said pressure sensing body is arranged to be advanced from said initial state to protrude from said outer sheath; and
a pressure sensor arranged at or adjacent to said distal part of said pressure sensing body for sensing pressure.

20. The implantable medical device according to claim 16, wherein said pressure sensing body comprises:
a tip section;
a first body section having a first cross-section area; and
a second body section having a second cross-section area, said second cross-section area being larger than said first cross-section area; and wherein said first and second body section are arranged such that a step is formed between said first and said second body section.

21. The implantable medical device according to claim 20, wherein said first and said second body sections are cylindrically shaped, wherein said second body section having a larger diameter than said first body section.

22. The implantable medical device according to claim 20, wherein said pressure sensor is integrated in said second body section.

23. The implantable medical device according to claim 20, wherein said step is shaped so as to form a cutting edge.

24. The implantable medical device according to claim 20, wherein said pressure sensor is integrated in said first body section.

25. The implantable medical device according to claim 16, wherein said distal part comprises a body section having a distal end shaped so as to form a cutting edge.

26. The implantable medical device according to claim 25, wherein said pressure sensor is integrated in said body section.

27. The implantable medical device according to claim 1, wherein said pressure sensing body comprises a threaded section arranged to mate with an inner threaded surface of said outer sheath, wherein said pressure sensing body is arranged to be advanced from said initial state to protrude from said outer sheath by a screwing motion.

28. An implantable sensor system connectable to a lead at a distal end of said lead, comprising:
an outer sheath;
at least one electrode located on said outer sheath for delivering pacing pulses to tissue;
a helically shaped needle arranged at said outer sheath, or in said outer sheath, wherein said helically shaped needle is substantially completely covered by said outer sheath at an initial state of said helically shape needle and wherein said helically shaped needle is arranged to be advanced from said initial state by a screwing motion to protrude from said outer sheath;
a pressure sensing body having a distal part and being movably arranged in said outer sheath, said pressure sensing body being arranged such that said distal part is located within said outer sheath in an initial state of said pressure sensing body, wherein said pressure sensing body is arranged to be advanced from said initial state to protrude from said outer sheath; and
a pressure sensor arranged at or adjacent to said distal part of said pressure sensing body for sensing pressure.

29. The sensor system according to claim 28, further comprising at least one sensor for sensing a physiological and/or hemodynamical parameter including impedance, blood temperature, heart rate, an activity level of a patient, oxygen level, or blood sugar level.

30. An implantable medical device for measuring pressure connectable to a medical lead, comprising:
an outer sheath;
a fixation mechanism; and
a pressure sensor for sensing pressure, the pressure sensor being movably arranged within the outer sheath, the pressure sensor being adapted to advance from a retracted position, at least partially within the outer sheath, to protrude from said outer sheath such that it is at least partially surrounded by said fixation mechanism in an extended position.
